(19) United States
(12) Patent Application Publication
(10) Pub. No.: US 2016/0081539 A1
(43) Pub. Date: Mar. 24, 2016

(54) MEDICO-SURGICAL VIEWING ASSEMBLIES, GUIDES AND INTRODUCERS

(71) Applicant: SMITHS MEDICAL INTERNATIONAL LIMITED, Kent TN25 4BF (GB)

(72) Inventor: Eric Pagan, Kent (GB)

(73) Assignee: SMITHS MEDICAL INTERNATIONAL LIMITED, Kent TN25 4BF (GB)

(21) Appl. No.: 14/889,202

(22) PCT Filed: Mar. 27, 2014
(86) PCT No.: PCT/GB2014/000114

§ 371 Date: Nov. 5, 2015

(30) Foreign Application Priority Data
May 11, 2013 (GB) 1308528.7

(51) Int. Cl. A61B 1/04; A61B 1/267; A61M 16/04; A61B 1/00

(52) U.S. Cl. CPC A61B 1/04; A61B 1/00045; A61B 1/267; A61M 16/0488; A61B 1/00027; A61B 1/00066

(57) ABSTRACT

A laryngoscope (10) includes a video camera ((13) at the patient end of its blade (11) that supplies video signals to a processor (14) in the handle (12). The processor (14) supplies signals to a display (15) supported on the handle (15). The laryngoscope also has a connector (17) on its handle (12) to which is connected the output of a video bougie guide (20) used with the laryngoscope. A switch (18) on the handle (12) enables the user to select which video output is shown on the display (15). Alternatively, images from both the bougie (20) and the laryngoscope (10) can be shown at the same time side-by-side on the display (15).

This invention relates to assemblies of the kind including an introducer and a guide, the guide being slidable along and removable from the introducer such that the guide and introducer can be used together to insert the guide into a body cavity so that a medical device can then be slid along the guide into the cavity and the guide subsequently removed to leave the device in the cavity.

The invention is more particularly concerned with viewing assemblies for use in placing a medical device within a patient such as inserting an endotracheal tube into the trachea. Traditional introducers or bougies take the form of a simple rod that can be bent to an approximate desired shape and can flex to accommodate the shape of the anatomy during insertion. The introducer may be made with an angled, Coudé tip to facilitate introduction. The introducer can be inserted more easily than the tube itself because it has a smaller diameter and can be bent and can flex to the ideal shape for insertion. The small diameter also gives the clinician a better view of the trachea around the outside of the introducer. These bougies may be used with or without the aid of a laryngoscope. When the bougie has been correctly inserted, a tube can be slid along its outside to the correct location, after which the bougie is pulled out of the tube, which is left in position. Bougies are available from Smiths Medical. GB2312378 describes an introducer or bougie moulded of an aliphatic polyurethane material and also describes an earlier bougie made from a braided polyester filament repeatedly coated in layers of resin. The characteristic flexural and recovery properties of these bougies are highly valued by clinicians.

More recently it has been proposed to use fibre optics or a video camera with an introducer to provide the clinician with a view of the trachea as the introducer is inserted. WO2010/136748 describes an introducer arrangement with a camera at one end of an introducer rod and a connector fixed to the opposite end of the rod by which the introducer can be connected via a cable to a display screen. The introducer is disconnected from the display when correctly positioned so that an endotracheal tube can be slid along the introducer into position, following which the introducer is removed by pulling rearwardly from the endotracheal tube. Other arrangements are described, for example, in WO2013/093391, WO2013/124606, WO2013/124605, GB2499714, GB2499708 and PCT/GB2013/000411.

Where the clinician uses a laryngoscope to introduce an endotracheal tube this may be of the kind including a video camera and a display screen, either attached to the laryngoscope or separate from it but mounted adjacent. Video laryngoscopes are available from McGrath, Glidescope, Storz, Intavent and other manufacturers. Examples of video laryngoscopes can be seen in U.S. Pat. No. 6,543,447, U.S. Pat. No. 5,827,178 and U.S. Pat. No. 5,800,344.

It is an object of the present invention to provide alternative medico-surgical viewing assemblies, guides and introducers.

According to one aspect of the present invention there is provided an assembly of the above-specified kind, characterised in that both the guide and the introducer include a video camera, and that the assembly includes a display on which images derived from the video camera in both the guide and the introducer can be presented.

The introducer is preferably a laryngoscope. The medical device may be an endotracheal tube. The guide preferably includes a bendable elongate member. The display is preferably supported on the introducer. The assembly may include a cable, the video camera in the guide being electrically connected to the display via the cable. The cable is preferably connected to a connector on a housing of the introducer, the connector on the housing being connected with a processor in the introducer, the processor providing an output to the display. Power for the video camera in the guide may be supplied from the introducer via the cable. The assembly may be arranged to present a representation on the display derived selectively from the video output of either the introducer or the guide. The introducer may include a manually-operable switch by which the display representation can be switched between the video output of the introducer and the guide. Alternatively the display may be arranged to present a display representation derived from the outputs of both the introducer and the guide at the same time in different regions of the display.

According to another aspect of the present invention there is provided an introducer for use in an assembly according to the above one aspect of the present invention.

The introducer may include a display supported on the introducer, the introducer including a processor for receiving an output from the video camera in the guide and an output from the video camera in the introducer, the introducer being arranged to provide outputs to the display representative of both the image from the video camera in the guide and the video camera in the introducer.

According to a further aspect of the present invention there is provided a laryngoscope including a handle, a blade extending from one end of the handle, a video device for providing a first output representative of a field of view towards the patient end of the blade and a display arranged to receive the output of the video device to provide a display representation of the field of view, characterised in that the laryngoscope is arranged to receive a second output from a video device in a guide used with the laryngoscope, and that the laryngoscope is arranged to provide a display representation on the display of the field of view of the video device in the guide as well as that in the laryngoscope either at the same time or separately.

The laryngoscope may include an electrical connector by which the second output is supplied to the laryngoscope. The display may be mounted on the handle of the laryngoscope.

An assembly of a laryngoscope introducer and endotracheal bougie guide will now be described, by way of example, with reference to the accompanying drawing, in which:

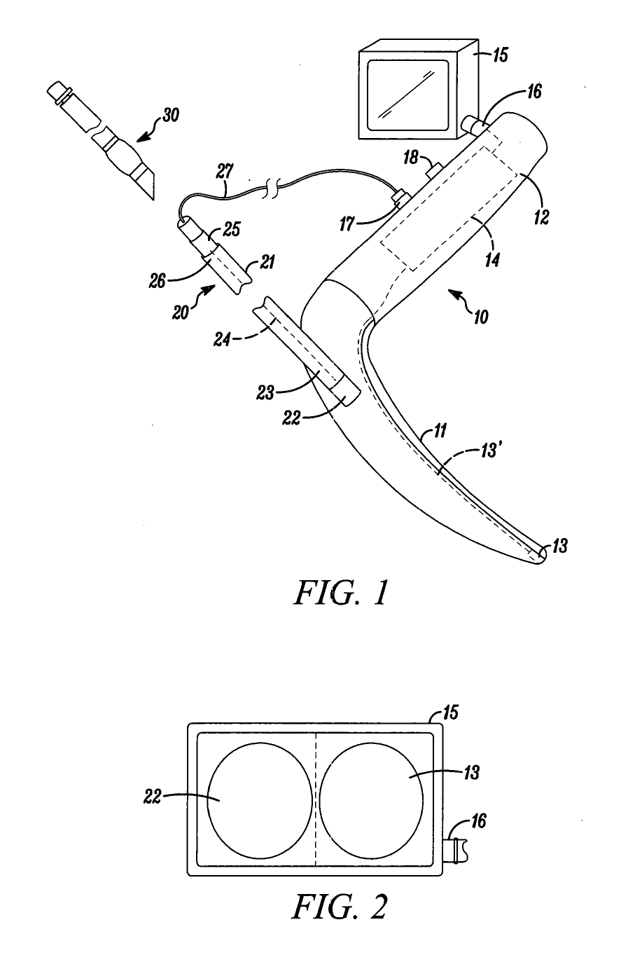

FIG. 1 is a perspective view of the assembly; and

FIG. 2 shows an example of a display representation.

The assembly comprises a video laryngoscope 10 and a video bougie 20. The assembly is used to introduce an endotracheal tube 30 into the trachea of a patient.

The laryngoscope 10 has a curved blade 11 mounted at one end of a handle 12. A video camera unit 13 is mounted on the blade 11 and is connected via a cable 13′ with a power supply and processor indicated generally by the numeral 14 in the handle 12. The output from the video camera 13 therefore provides a first output supplied to the processor 14. The video camera unit 13 includes a conventional video chip and illumination means such as one or more LEDs. Alternatively, the video camera unit could be mounted in the handle 12 and connected to an optical fibre light guide extending to the patient end of the blade 11. The rear end of the handle 12, remote from the blade 11, supports a display panel 15 mounted on a pivotal joint 16 that enables the panel to be angled as desired by the user. The display panel 15 is electrically connected to the processor 14, which provides signals to drive the display panel to provide a representation of the field of view of the video camera unit 13. As so far described the laryngoscope 10 is conventional. The laryngoscope 10 differs from conventional laryngoscopes in having means for receiving a video input from a second source, that is, from the video bougie 20. In particular, the handle 12 includes an electrical connector 17 on its outer housing connected internally of the handle with the processor 14. The laryngoscope 10 also includes a manually-operable control 18 such as a button or switch by which the user can select whether to show an image derived from the laryngoscope or from the bougie 20 in a manner that will be described in detail later.

The video bougie 20 includes an elongate member in the form of a bendable rod 21 of a plastics material having a video camera unit 22 mounted at its patient end 23 and connected by a cable 24 extending along the length of the rod to an electrical connector 25 fixed on the rear, machine end 26 of the rod. The camera unit 22 includes a conventional video camera chip and illumination means such as one or more LEDs. In conventional use, the connector 25 at the rear end of the bougie 20 would be connected to a dedicated display unit via a cable. In the present invention, by contrast, the connector 25 is connected instead via a cable 27 to the connector 17 on the handle 12 of the laryngoscope 10. The output of the camera 22 in the bougie 20, therefore, provides a second output supplied to the processor 14 so that a display representation of the field of view of the bougie camera can be presented on the display panel 15. Similarly, power to drive the illumination means in the bougie camera unit 22 is derived from the processor and power supply unit 14 in the laryngoscope 10 via the cable 27. By actuating the control 18, the user can switch between viewing an image from the camera 22 in the bougie 20 and the camera 13 in the laryngoscope blade 11.

Instead of selectively switching manually between the different camera outputs it would be possible to switch automatically, such as under control of a timer. Alternatively, it would be possible to display images from both cameras 13 and 22 on the display panel 15 at the same time in different regions of the display, such as side-by-side in a split screen layout illustrated in FIG. 2.

The bougie could include a wireless transmission unit such as infra-red or RF, such as using Bluetooth protocol. The transmitter could either be built into the bougie, such as in a handle portion at the rear end, or it could be provided as a separate component connected removably to the connector 25. The laryngoscope would then include a wireless receiver arranged to receive the wireless signals from the bougie. In wireless arrangements of this kind the guide would require an independent power source, such as from its own battery.

In the arrangement illustrated in FIG. 1, the bougie 20 has a low profile connector 25 at its rear end that enables the endotracheal tube 30 to be loaded after the bougie has been positioned correctly in the patient, simply by disconnecting the cable 27 and sliding the tube onto the bougie from its rear end over the connector. The guide, however, could instead have an enlarged handle at its rear end (such as for containing a wireless transmitter and power supply) in which case, the endotracheal tube would be preloaded on the guide from the patient end of the guide before it is assembled with the laryngoscope.

The present invention enables the amount of equipment needed around the patient to be reduced and allows the anaesthetist to view the output of both the laryngoscope and bougie at the same location, thereby facilitating intubation.

The introducer need not be a laryngoscope but could be some other form of introducer for use in inserting a medical device into a body cavity.

1-16. (canceled)

17. An assembly of an introducer and a guide, the guide being slidable along and removable from the introducer such that the guide and introducer can be used together to insert the guide into the body cavity so that the medical device can then be slid along the guide into the cavity and the guide subsequently removed to leave the device in the cavity, characterized in that both the guide and the introducer include a video camera, and that the assembly includes a display on which images derived from the video camera in both the guide and the introducer can be presented.

18. An assembly according to claim 17, characterized in that the introducer is a laryngoscope.

19. An assembly according to claim 17, characterized in that the medical device is an endotracheal tube.

20. An assembly according to claim 17, characterized in that the guide includes a bendable elongate member.

21. An assembly according to claim 17, characterized in that the display is supported on the introducer.

22. An assembly according to claim 17, characterized in that the assembly includes a cable, and that the video camera in the guide is electrically connected to the display via the cable.

23. An assembly according to claim 22, characterized in that the cable is connected to a connector on a housing of the introducer, that the connector on the housing is connected with a processor in the introducer, and that the processor provides an output to the display.

24. An assembly according to claim 23, characterized in that power for the video camera in the guide is supplied from the introducer via the cable.

25. An assembly according to claim 17, characterized in that assembly is arranged to present a representation on the display derived selectively from the video output of either the introducer or the guide.

26. An assembly according to claim 25, characterized in that the introducer includes a manually-operable switch by which the display representation on the display can be switched between the video output of the introducer and the video output of the guide.

27. An assembly according to claim 17, characterized in that the display is arranged to present a display representation derived from the outputs of both the introducer and the guide at the same time in different regions of the display.

28. An introducer for use in an assembly having an introducer and a guide, the guide being slidable along and removable from the introducer such that the guide and introducer can be used together to insert the guide into a body cavity so that the medical device can then be slid along the guide into the cavity and the guide subsequently removed to leave the device in the cavity, wherein both the guide and the introducer include a video camera, and wherein the assembly includes a display on which images derived from the video camera in both the guide and the introducer can be presented.

29. An introducer according to claim 28, wherein the display is supported on the introducer, characterized in that the introducer includes a processor for receiving an output from the video camera in the guide and an output from the video camera in the introducer, and that the introducer is arranged to provide outputs to the display representative of both the image from the video camera in the guide and the video camera in the introducer.

30. A laryngoscope including a handle, a blade extending from one end of the handle, a video device for providing a first output representative of a field of view towards the patient end of the blade and a display arranged to receive the output of the video device to provide a display representation of the field of view, characterized in that the laryngoscope is arranged to receive a second output from a video device in a guide used with the laryngoscope, and that the laryngoscope is arranged to provide a display representation on the display of the filed of view of the video device in the guide as well as that in the laryngoscope either at the same time or separately.

31. A laryngoscope according to claim 30, characterized in that the laryngoscope includes an electrical connector by which the second output is supplied to the laryngoscope.

32. A laryngoscope according to claim 30, characterized in that the display is mounted on the handle of the laryngoscope.
